(19) United States
(12) Patent Application Publication
(10) Pub. No.: US 2016/0206316 A1
(43) Pub. Date: Jul. 21, 2016

(54) SUTURELESS ANASTOMOSIS DEVICE

(71) Applicants: Cameron L. Johnson, Salt Lake City, UT (US); Carolyn Johnson, Salt Lake City, UT (US); Adam Leffler, Salt Lake City, UT (US)

(72) Inventors: Cameron L. Johnson, Salt Lake City, UT (US); Carolyn Johnson, Salt Lake City, UT (US); Adam Leffler, Salt Lake City, UT (US)

(21) Appl. No.: 14/598,185

(22) Filed: Jan. 15, 2015

(51) Int. Cl. A61B 17/11

(52) U.S. Cl. CPC A61B 17/11; A61B 2017/1107; A61B 2017/1132

(57) ABSTRACT

Innovative and effective snap connector for sutureless vascular anastomosis is disclosed. The jas is made of material that can be absorbed by the human body which promotes faster and more effective healing, has three simple and easy to use parts, and takes on average less than two minutes to assemble. Use of the jas is effective at obtaining a proper seal, reducing potential complications that can arise from long operation times or more complicated application systems, reduces time in surgery, and reduces time for patient's to heal post-surgery.

### CROSS-REFERENCED APPLICATION

This application claims priority to U.S. Provisional Patent Application 61/927,873 filed Jan. 15, 2014.

### BACKGROUND OF THE INVENTION

The current use of sutures to complete vascular anastomosis is time consuming, which can lead to exposing a patient to additional bacteria in the air during surgery. This can lead to an increased time for recovery. The current practice of suture focused end-to-end vascular anastomosis is imperfect and prone to leakage which requires additional blood, more doctor's time, more time in the operating room, and extends costs to patients and insurance companies. Expense driven partially by current practice leads to some patients not being able to afford or receive necessary life-improving or life-saving operations.

### SUMMARY OF THE INVENTION

The current use of sutures to complete vascular anastomosis requires an average of 20 minutes per connection, which can lead to exposing a patient to bacteria in the air during surgery. A lengthy time during surgery weakens the patient's health, and creates a lengthy time for healing. The current practice of suture focused end-to-end vascular anastomosis is imperfect and prone to leakage which requires additional blood, more doctor's time, more time in the operating room, and extends costs to patients and insurance companies. Expense driven partially by current practice leads to some patients not being able to afford or receive necessary life-improving or life-saving operations. Problems related to the current practice can also cause complications including death.

The present invention takes on average under two minutes to complete sutureless end-to-end vascular anastomosis, saving an average of 18 minutes per connection during any surgery. Healing time is reduced significantly. Use of the sutureless end-to-end vascular anastomosis significantly reduces the likelihood of leakage. The cumulative benefit reduces patient time in the operating room, reduces medical staff time in operating room, opens up more time for additional surgeries or other uses of operating rooms generally, reduces costs to patients, and reduces costs to insurance companies. Considering a combination of time savings and cost savings, more patients will arguably be able to afford and receive necessary life-improving or life-saving operations.

### BRIEF DESCRIPTION OF THE DRAWINGS

FIG. 1 is an elevational view of a key;

FIG. 2 is a cross-sectional view of a key;

FIG. 3 is a perspective view of a key;

FIG. 4 is an elevational view of an inside bell;

FIG. 5 is a cross-sectional view taken along lines A-A in FIG. 4 of an inside bell;

FIG. 6 is a perspective view of the inside bell;

FIG. 7 is an elevational view of an exterior bell;

FIG. 8 is a cross-sectional view taken along lines A-A in FIG. 7 of an exterior bell;

FIG. 9 is a perspective view of the exterior bell shown in FIG. 7;

FIG. 10 is an elevational view of a locking ring;

FIG. 11 is a cross-sectional view of a locking ring;

FIG. 12 is a perspective view of a locking ring;

FIG. 13 is a view of the three pieces prior to assembly; and

FIG. 14 shows the three pieces assembled on a vein;

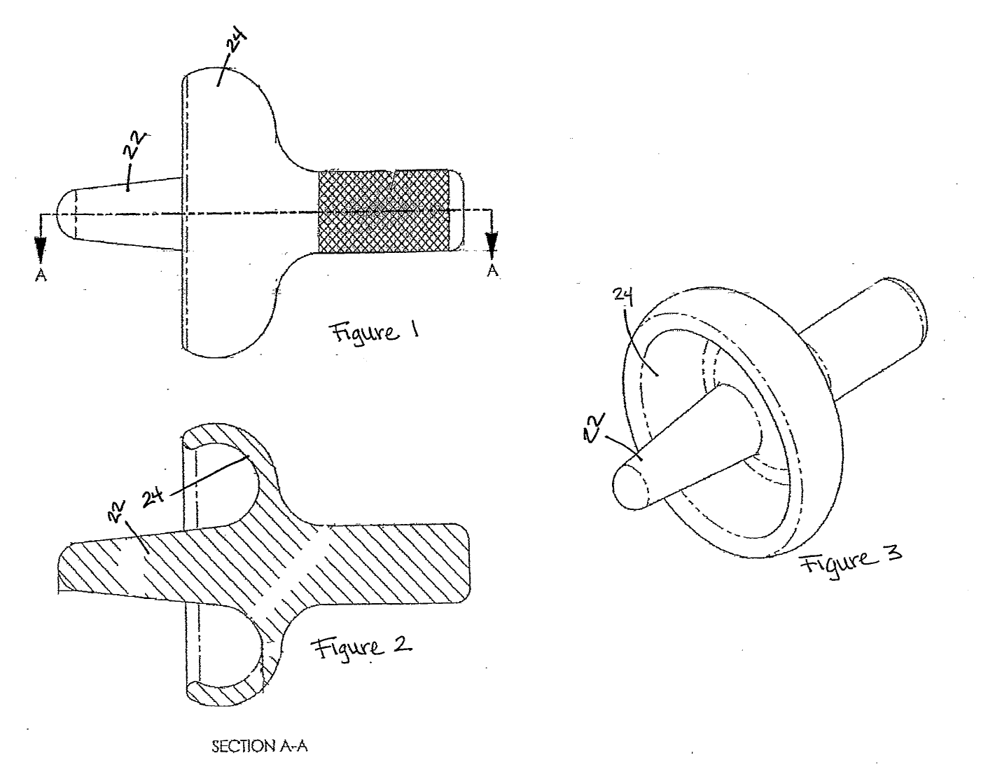

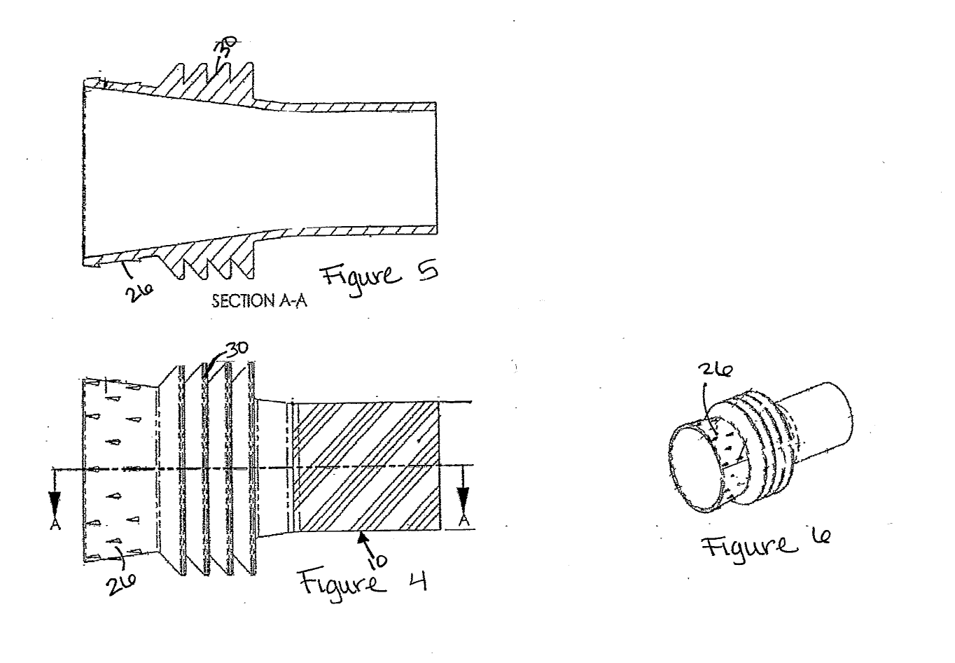

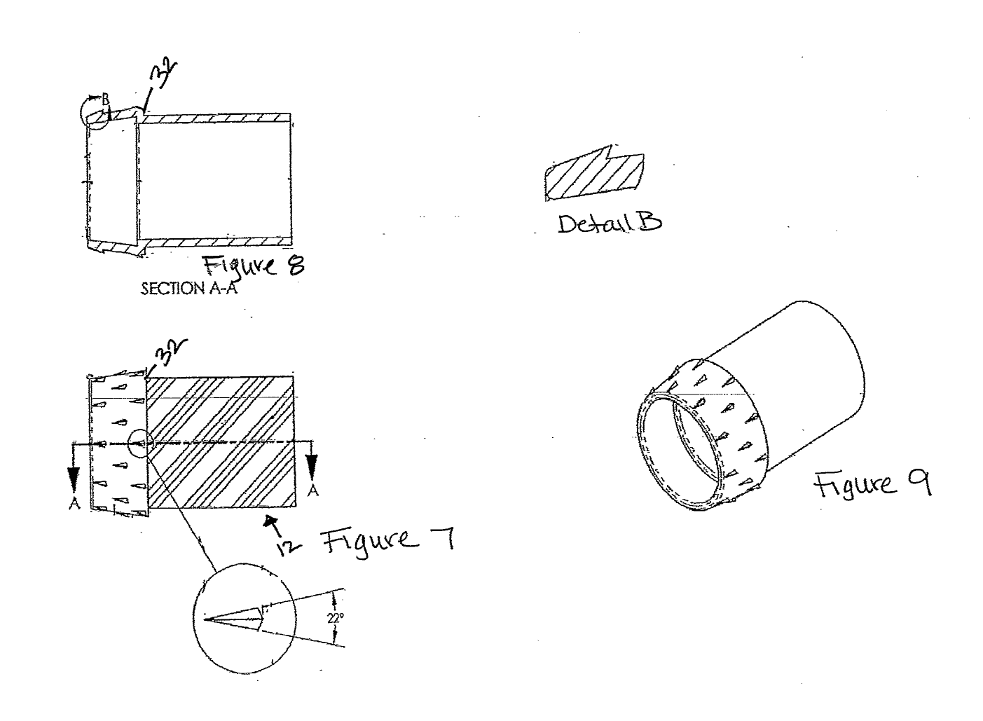

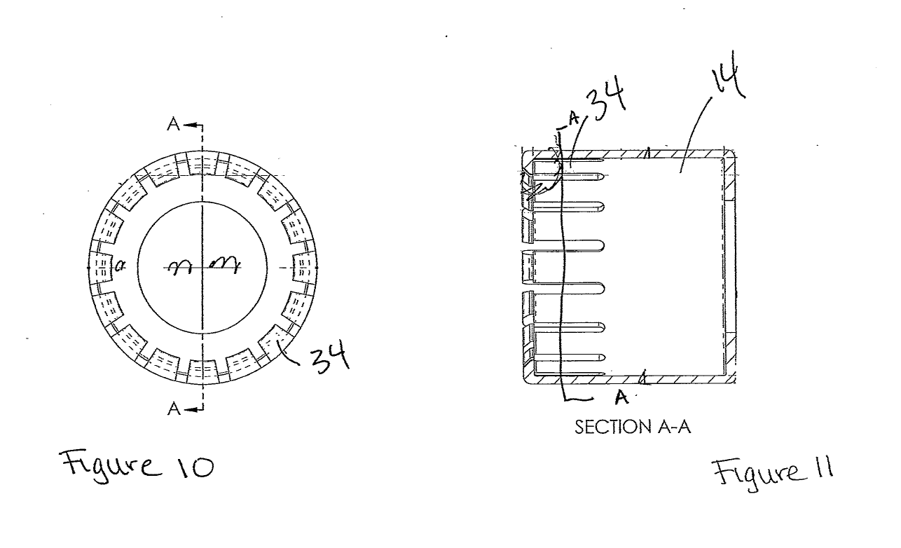

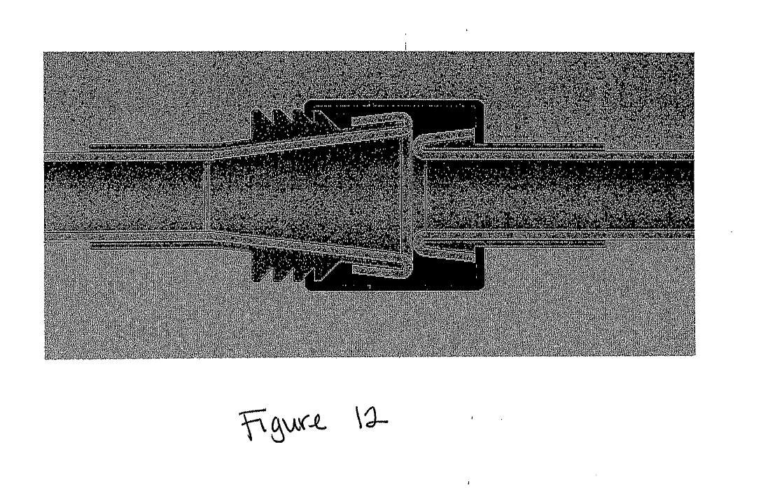

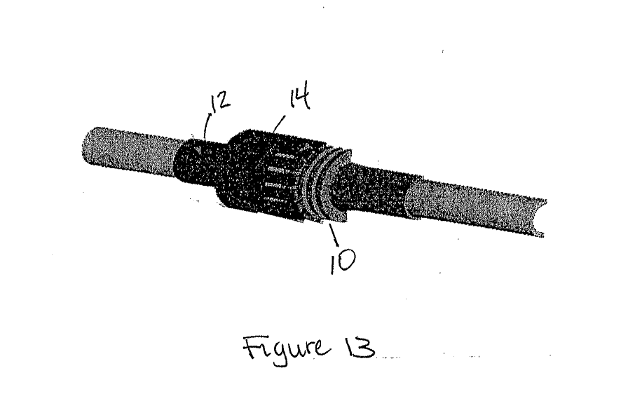

### DETAILED DESCRIPTION OF THE PREFERRED EMBODIMENTS

Other current sutureless end-to-end anastomosis devices have more parts, are more complicated, and are made of materials that are not absorbable by the human body. The present invention consists of only three simple parts that are more easily and speedily assembled, made with materials that are absorbable by the human body, and does not use materials such as glue which can pose additional problems for a patient.

The present invention is made of material that can be absorbed by the human body and does not therefore need to be removed. This promotes faster and more effective healing. The preferred embodiment has three simple and easy to use parts, and takes on average less than two minutes to assemble.

The preferred embodiment of the present invention comprises three pieces when assembled. An exterior bell 10, and interior bell 12, and a locking ring 14. A key 16 is used to slide the vein over barbed ends 18 and 20 of exterior bell 10 and interior bell 12.

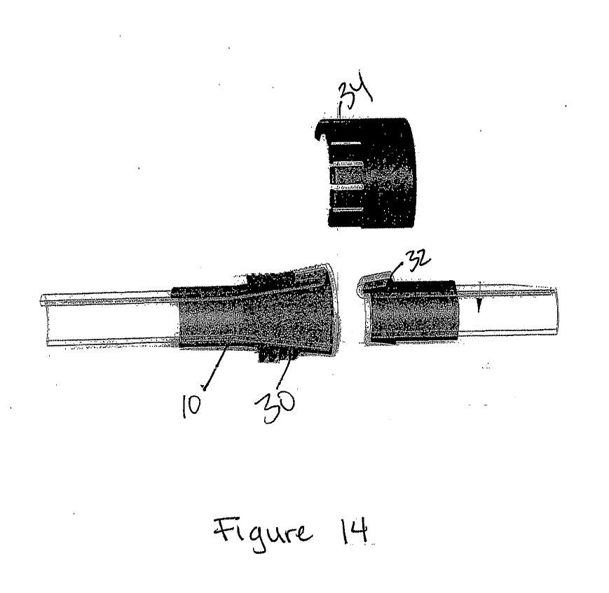

Referring now to the Figures, a key 16 is shown. Key 16 has an expander 22 which is inserted into the open end of a severed vein or artery. As expander 22 is pressed further into the end of a central vein, the vein diameter is increased and further pressing of key 16 into the vein results in the vein being expanded and bent back as the vein encounters curved wall 24. By placing exterior bell 10 over an end of a severed vein or artery and extending the vein through the bell so that a portion of the vein extends beyond the end of the bell, key 16 may be used by the other hand of the surgeon to expand the vein and bend it back over itself so that expander 22 may be inserted into the end of a barbed portion 26 of the end of exterior bell 10. The vein can then be slid off of the expander 22 and on to the barbs so that the vein is retained on exterior bell 10. Locking ring 14 will have already been placed on interior bell 12 before the vein is attached. In some embodiments, a split locking ring may be used that does not require prior placement. Key 16 is now used to expand the other end of the severed vein in the same manner and is used to locate the portion of the vein extending out of the end of interior bell 12 on to a barbed portion 28 of interior bell 12. Each end of the vein is now attached to a bell. Exterior bell 10 has a larger diameter and is sized so that the end of interior bell 12 will slid into the end of exterior bell 10. When the two bells are interconnected, locking ring 14 is pushed and snapped over ribs 30 on the outside of exterior bell 10 until locking ring 14 encounters a shoulder 32 on interior bell 12. The locking ring 14 is tightened only enough to ensure that there will be no leakage. Teeth 34 on locking ring 14 engage ribs 30 on the outside of exterior bell 10 to lock the two bells together in positive engagement thereby completing the sutureless end-to-end vascular anastomosis. FIG. 13 shows the exterior bell 10, interior bell 12 and locking ring 14 prior to assembly and FIG. 14 shows the three elements in their assembled formation on a vein.

What is claimed is:

1. A sutureless end-to-end vascular anastomosis device, comprising:
an exterior bell having a receiving end for receiving an end of a severed vein, a barbed end for retaining the end of the vein and on the outer surface, ribs;
an interior bell having a receiving end for receiving an end of a severed vein, an opposing barbed end for retaining the vein and on the outer surface a shoulder; and
a locking ring having teeth to engage the ribs on the outer surface of the exterior bell and he body of the locking ring being configured to engage the shoulder of the interior bell, so that when the locking ring is pressed over the interior bell the teeth of the locking ring engage the ribs on the exterior bell to lock the bells together.

2. The sutureless end-to-end vascular anastomosis device as recited in claim 1, wherein the barbed end of the interior bell is sized to fit the barbed end of the exterior bell.

3. A sutureless end-to-end vascular anastomosis device as recited in claim 1, wherein the device is constructed of a material that is absorbable by the human body.

4. A sutureless end-to-end vascular anastomosis device as recited in claim 1, wherein the locking ring is split.

5. A method for performing sutureless end-to-end vascular anastomosis, comprising the steps of:
placing a locking ring over an interior bell;
inserting a severed vein into a receiving end of an interior bell;
inserting the other end of a severed vein into the receiving end of an exterior bell;
using a key to expand the vein and place it over a barbed in of the interior bell;
using a key to expand the vein and place it over a barbed end of the exterior bell;
inserting the barbed end of the interior bell into the barbed end of the exterior bell; and
gliding the locking ring over the interior bell until teeth on the locking ring engage ribs on the exterior bell to lock the interior bell into positive engagement with the exterior bell.
